(19) United States
(12) Patent Application Publication
(10) Pub. No.: US 2002/0055118 A1
(43) Pub. Date: May 9, 2002

(54) Method of preparing objects containing DNA

(76) Inventor: Yong-Bin Eym, Kimpo-City (KR)

Correspondence Address: JACOBSON HOLMAN PLLC, 400 SEVENTH STREET N.W., SUITE 600, WASHINGTON, DC, 20004, US

(21) Appl. No.: 09/987,005

(22) Filed: Nov. 13, 2001

Related U.S. Application Data
(63) Continuation-in-part of application No. 09/720,332, filed on Dec. 22, 2000.

(30) Foreign Application Priority Data
Jun. 24, 1998 (KR) 1998-23810

(51) Int. Cl. C12Q 1/68; B32B 23/04

(52) U.S. Cl. 435/6; 427/2.11; 428/532; 442/59

(57) ABSTRACT

A method of preparing an object containing DNA, the method comprising the steps of: obtaining a sample including DNA from a donor; extracting DNA from the sample; amplifying the extracted DNA; introducing a solution of the amplified DNA into a cavity within the object through an opening of the cavity, and closing the opening of the cavity, wherein the object is with a defined shape, or mixing a solution of the amplified DNA with the object where the object is amorphous, and a method for producing an object containing DNA, comprising the steps of: image-scanning a polyacrylamide gel film containing electrophoresed DNA bands; offset-printing on an object having a fixed shape using the scanned image data; and applying a DNA solution onto the DNA band image which has been offset-printed.

This application is a continuation-in-part application of patent application Ser. No. 09/720,332, filed Dec. 22, 2000, the contents of which are incorporated herein by reference.

### FIELD OF THE INVENTION

The invention relates to a method of preparing an object containing DNA.

### BACKGROUND OF THE INVENTION

DNA is a basic generic material contained in chromosomes of cell organelles. Since DNA stores genetic information, it has been the subject of extensive study in both biological and medical fields. However, since only a small amount of DNA can be obtained from a living organism, the target sequences of DNA must be amplified before the DNA can be studied. Various methods and apparatuses for amplifying target sequences of DNA are known. (Mullis, K. et al, Specific enzymatic amplification of DNA in vitro: the polymerase chain reaction., Cold Spring Harbor Symp. Quant. Biol. 51, 263-273, 1986; U.S. Pat. Nos. 4,683,195, 4,683,202, and 4,889,818, Korean Patent No. 126,231)

The inventors have noted, in view of the fact that DNA can uniquely represent differentiated characteristics of a donor, that objects that have excellent durability and appearance can be obtained by injecting DNA into or mixing DNA with various objects, or by applying a film-like dried gel to the objects, where there are needs for carrying a character of the DNA donor with the object. The objects thus prepared represent the donor of the DNA by embodying the differentiated characteristics of the donor.

Also, the method of applying a film-like dried gel directly onto the object has such problems as variations between mass-produced objects and the high cost due to the need that individual steps for DNA extraction, amplification, electrophoresis and staining are required for each object. The present invention also addresses these problems.

### SUMMARY OF THE INVENTION

An object of the present invention is to provide a method of preparing solid objects, such as accessories, records and stationaries that contain DNA by injecting DNA of a donor into the objects and a method of preparing amorphous objects, such as perfume and ink, which contain DNA by mixing DNA of a donor with the objects. Both methods employ DNA extraction and amplification techniques.

Also, another object of the present invention is to provide a method for producing objects containing DNA on DNA band images, with low cost and without variations between products.

The present invention also provides objects containing DNA prepared by any one of the methods described above.

According to the first embodiment of the invention, there is provided a method of preparing a solid object containing DNA, the method comprising the steps of: obtaining a sample including DNA from a donor; extracting DNA from the sample; amplifying the extracted DNA; introducing a solution of the amplified DNA into a cavity within the object through an opening of the cavity; and closing the opening of the cavity.

Alternatively, the solution of amplified DNA may be dried into a gel or solid state and the dried DNA introduced into the cavity of the solid object.

According to the second embodiment of the invention, there is provided a method of preparing an amorphous object containing DNA, the method comprising the steps of: obtaining a sample including DNA from a donor; extracting DNA from the sample; amplifying the extracted DNA; and mixing a solution of the amplified DNA with the object.

Alternatively, the solution of amplified DNA may be dried into a gel or solid state and the dried DNA mixed with the amorphous object.

According to the third embodiment of the invention, there is provided a method of preparing a solid object containing DNA, the method comprising the steps of: obtaining a sample including DNA from a donor; extracting DNA from the sample; amplifying the extracted DNA; subjecting the amplified DNA to a electrophoresis in a polyacrylamide gel; dyeing the electrophoresed DNA; drying the gel containing the dyed DNA to obtain a gel film; and applying the gel film to a surface of the object.

According to the fourth embodiment of the invention, there is provided a method of preparing a solid object containing DNA, the method comprising the steps of: obtaining a sample including DNA from a donor; extracting DNA from the sample; amplifying the extracted DNA; preparing HLA DNA band strips with the amplified DNA; and applying the HLA DNA band strips to a surface of the object.

According to the fifth embodiment of the present invention, there is provided a method for producing an object containing DNA, comprising the steps of: image-scanning a polyacrylamide gel film containing electrophoresed DNA bands; offset-printing on an object having a fixed shape using the scanned image data; and applying a DNA solution onto the DNA band image which has been offset-printed.

### BRIEF DESCRIPTION OF THE DRAWINGS

FIG. 1 shows a key ring that contains a DNA mixture prepared according to an embodiment of the present invention, and HLA DNA band strips.

FIG. 2 shows a necklace prepared according to another embodiment of the present invention.

FIG. 3 shows a necklace prepared according to a yet another embodiment of the present invention.

FIG. 4 shows the result of a DNA analysis for the DNA containing object produced in Example 3.

FIGS. 5 and 6 schematically show the front and back sides, respectively, of a card produced according to still another embodiment of the present invention.

In the figures, each numeral has the following meaning:

1: a key ring

2: HLA DNA band strips

3: picture of the donor of DNA

4: silk-screen signature

5: solution containing DNA

6: water

10, 20: necklaces

31: picture of a celebrity

32: name of the celebrity

33: date of birth

34: height of the celebrity

35: blood type

36: genotype

37: hologram foil

38: DNA bands

39: serial number

40: picture of a celebrity

41: logo of the manufacturer

42: signature box

### DETAILED DESCRIPTION OF THE INVENTION

The donor of DNA can be any living organism having DNA including a human being. In view of the demand for objects, celebrated figures associated with celebrities, such as political leaders, religious leaders, authors, movie stars, sports stars and singers, would be preferable donors of DNA. However, family members, members of a group, or pets could also be donors, since objects associated with these donors would be valuable to the other family members, group members, or pet owners, respectively.

The DNA sample may be any part or specimen that can be collected from the donor and contains the DNA of the donor. For a human DNA donor, blood, hair, skin tissue, fingernail, toenail, etc. may conveniently be used as the DNA sample.

DNA can be extracted and recovered by various known methods, though the methods could be modified depending on the DNA sample to be used (Buffone et al, Isolation of DNA from biological specimens without extraction with phenol. Clin. Chem. 31: 164-165, 1985). If, for example, the sample were blood, EDTA blood and RBC lysis buffer solution would be mixed with the sample and the mixture would be centrifuged. The residue thus obtained would be mixed with Chelex resin and boiled to extract DNA.

First, the first to fourth embodiments of the invention will be described.

Natural genomic DNA may be used for preparing objects containing DNA in accordance with the present invention. However, since only a small amount of natural DNA would be extracted from a sample, it is preferable to amplify the extracted natural DNA.

DNA amplification may be carried out by polymerase chain reaction(s), or by two-stage amplifications, where the first amplification stage comprises a polymerase chain reaction, and the second amplification stage comprises the steps of inserting the DNA amplified in the first stage amplification into a vector to obtain a recombinant plasmid, introducing the recombinant plasmid into a host cell, culturing the host cell transformed by the recombinant plasmid, and separating the plasmids thus produced in large amounts.

The polymerase chain reaction (PCR) may be performed according to a method of Mullis et al., by means of a conventional polymerase chain reaction apparatus. Gene Kit (HLA Genotyping kit, Inno-lipa Co., Ltd.) may be conveniently used for the amplification procedure.

In the second amplification stage using plasmids, the DNA amplified by the PCR may be cloned, for example, by using a TA cloning kit (Invitrogen). Otherwise, the amplified DNA may be reacted without dNTP in a medium containing T4 DNA polymerase for 15 minutes, and then reacted with dNTP for 15 minutes to obtain blunt ended DNA, where T and A bases protruding from each end of the DNA have been removed. Separately, an appropriate vector to be used in cloning process is cleaved by using a blunt end restriction enzyme such as SmaI. To the vector thus obtained the blunt ended DNA is ligated by T4 DNA ligase.

The plasmid thus constructed may be introduced into an appropriate host cell for separating recombinant plasmids in large amounts. The host cell may be E. coli HB101, E. coli CSH 41, etc. The host cell transformed by the plasmid according to the present invention is cultured in Luria Broth (LB) medium, and then the plasmid produced in large amounts may be extracted and separated by a method of Birnboin & Doly (1979).

The resulting DNA solution may be injected into a cavity within a solid object through an opening of the cavity, and the opening of the cavity may be closed to obtain an object containing DNA. Optionally, the DNA solution may be dried into a gel or solid state, and the dried DNA introduced into the cavity of the solid object. The DNA solution or dried DNA may be dyed to make it visible before it is introduced into the object. The DNA solution may be dyed with a dyeing reagent, such as bromophenol blue or phenol red and optionally mixed with glycerol, before being injected into the object.

In the specification and claims, a “solid” object is any object having a defined shape, which may include, but is not limited to, jewelry such as rings, necklaces, ear rings, writing implements such as pens, fountain pens, pencils, pencil cases, and key rings, dolls, toys, shoes, bags, purses, wallets, records, CDs.

The cavity of the object into which DNA is to be injected may be formed by conventional processes. In order to enhance the decorative effect realized by dyed DNA, the cavity of the solid object preferably would be formed within a transformed pattern. The transparent portion may have various shapes. The object containing DNA according to the present invention may be further embellished with a picture of the double stranded DNA helix, a signature and/or a picture of the donor of DNA to indicate that the object contains DNA of the donor.

The amplified DNA solution may optionally be dried, before being mixed with an amorphous object. An “amorphous” object is any object not having a defined shape, which may include, but is not limited to perfume, ink, Chinese ink, and dyestuffs.

In accordance with an embodiment of the present invention, the amplified DNA may be subjected to electrophoresis in a polyacrylamide gel, dyed, and dried to obtain a gel film containing DNA. The resulting gel film may be applied to a surface of a solid object.

When DNA is extracted from a blood sample, HLA DNA band strips may be applied to a surface of the object to be decorated. Specific characteristics of the DNA donor can thus be exhibited by the HLA DNA band strips.

The HLA DNA band strips may be prepared as follows. DNA is extracted from a blood sample and the extracted DNA is amplified. The amplified DNA may be reacted with, for example, a kit strip available from Inno-lipa Co., Ltd. Since probes for identifying various DNA sequences of leukocytes are present on the strip, these probes combine with the DNA by making base pairings. If a dyeing reagent which can react with the DNA were added to the strip, the dyeing reagent would react with DNA combined with the probe to form a color. The band strips would vary depending on the donor of DNA, since each donor has his or her own specific DNA base sequences.

Since objects prepared according to the methods of the present invention contain DNA, which represent specific characteristics of the DNA donor, one may remember the donor, such as celebrated figures, family members, lovers, pets, etc., by carrying the objects with one.

Further, objects prepared according to the methods of the present invention may be distinguished objects from which do not contain DNA. The method of the present invention may be used for determining whether a certain object is genuine or counterfeit.

Next, the fifth embodiment of the invention will be described.

Film-like polyacrylamide gels containing electrophoresed DNA bands can be prepared by the method described above.

Specifically, a sample containing DNA is obtained from a donor, and the DNA is extracted from the sample. The extracted DNA is amplified, and electrophoresed on a polyacrylamide gel. The gel is then dried after staining.

PCR equipments can be used for the amplification of DNA, and the PCR may be carried out with polymorphic markers. In this case, parts of nucleotide sequences of the polymorphic markers may be utilized as the nucleotide sequences of the primers for the PCR. The size of the amplification products may vary depending on the polymorphic markers used. The amplification products may be subjected to electrophoresis on a polyacrylamide gel and silver staining, and their genotype can be identified by measuring the number of their repeating sequence. Thus, an object according to the present invention may carry an identification of the DNA donor's genotype.

Such genotype, which represents the number of repeating core for a polymorphic marker, means the genetic composition of an individual resulting in the individual's phenotype and differs between any two individuals. Therefore, the genotype can be an identifier for individuals. Various genotypes can be defined. In the Examples of the present application, the genotype was defined after amplifying DNA with 4 kinds of polymorphic markers. These markers were selected for their similarity in the size of the amplification products, which enables the desired DNA bands to be located within the defined gel size during electrophoresis, and suitable polymorphic markers are not limited thereto.

DNA polymorphisms are found even in the genomes of individuals of the same species. Fingerprinting is a genetic analysis technique using such polymorphisms, and currently utilized in various fields including paternity and forensics. Fingerprinting using PCR is adopted when the amount of the specimen is very small or the specimen is damaged. For example, PCR-based fingerprinting was utilized in identifying the bones of St. Nicholas II, the last Russian Tsar, and his family. Regions of polymorphism are given names to generate various polymorphic markers, and there are many polymorphic markers available (e.g., D17S695, D21S11, NeuR, YNZ22, etc.). Four kinds of such polymorphic markers (i.e., D17S695, D21S11, hTPO, humthol) were used in the Examples described herein. D17S695 is a locus present on chromosome No. 17 and includes 13 tandem repeats of the tetranucleotide ‘GAAA’. The normal size of the PCR product is 201 bp. D21S11 is present on chromosome No. 21 and includes 10 repeats of ‘ATCT’. The size of the PCR product is 206 bp. hTPO is present on chromosome No. 2, includes 11 repeats, produces a PCR product of 217 bp, and synthesize a thyroid peroxidase. humthol has 9 repeats of ‘CATT’ and produces a PCR product of 158 bp. It encodes a tyrosine hydroxylase. Although polymorphic markers may encode certain proteins, most of them are minisatellite DNAs which do not. Their functional roles have not been elucidated clearly, and it has been recently proposed that they may provide binding sites for transcription factors or may be involved in the structure of chromosomes.

Using a computer, image scans are collected from gels in a film-like form. Image-scanning refers to the copying of colors from an image using computerized color separation. Data from the image scans are processed to produce a printable offset film, which is normally C, M, Y, K 4 color-based: C for cyan, M for magenta, Y for yellow, and K for black. The processed image is then offset-printed at a suitable location on an object having a defined shape, for example, a plastic card (size: 8.6×5.5 cm). Since the printed substrate cannot be laminated with a PVC clear substrate, a special adhesive should be applied to the printed surface for lamination. Suitable special adhesive includes CDA02 from Sericol, England and 4684 from Apollo, USA.

Over the offset-printed DNA bands, suitable amount of a DNA solution is applied. A DNA solution prepared by obtaining a sample containing DNA, extracting and amplifying DNA from the sample can be used. Particulars about the preparation of DNA are described in Korean Laid-open publication No. 99-78599.

For the application of the DNA solution, the DNA solution is diluted with a special adhesive (from Sericol, Apollo, etc.). A gauze stencil is set up on a silk screen printer (adsorber), and the solution is diluted with a silk screen printing ink (a clear ink), applied using a squeegee (silk screen printing surface treatment), and then dried using drying equipments. A hologram foil is then covered over the DNA applied region, if appropriate when decoration is desired during the manufacturing of PVC cards or the like. Hologram foil refers to a synthetic resin substrate to which a special mineral matter (which has been made heat transferable to PVC, paper or other material by degradation of normal mineral matter) is printed and adhered in a desired shape and which can be combined to PVC, paper or other substrate by heat and pressure.

The objects containing DNA according to the present invention may be objects of toy, accessory, decorations, records, stationery, packaging paper, packages, sports gears, shoes, bags, clothes, cards, photos, souvenirs, art, funeral implements, wedding implements, or religious ceremony. In particular, production of various cards carrying DNA according to the present invention is advantageous.

The DNA contained in the objects prepared according to the methods of the present invention may be preserved without substantial change for long periods of time. Therefore, the DNA may be used to confirm a person's identity: i. e. the DNA contained in an object prepared according to a method of the present invention may be compared with the DNA obtained from a person to be identified, thereby to determine, if required, whether the person is the donor or, is genetically related to the donor.

According to the present invention, one may prepare various objects containing DNA. These objects can represent differentiated characteristics of the donor of the DNA and be preserved for a long time.

The method according to the present invention can provide, at low cost, durable objects containing DNA, which can represent differentiated characteristics of the DNA donor and can be kept intact for a prolonged time.

The following examples are provided to illustrate the method of preparing an object containing DNA according to the present invention.

### EXAMPLE 1

Extraction of DNA

3 ^{ml} of whole blood collected from a donor were placed into a EDTA coated tube, and at 2-8° C. in a refrigerator.

50 ^{μl} of EDTA blood and RBC lysis buffer solution were placed into a sterilized 1.5 ^{ml} micro-centrifuge tube and centrifuged at 13,000 rpm for one minute to obtain a precipitated residue. The residue was washed with 500 ^{μl} of sterilized water. The centrifugation and the washing steps were repeated until all remaining RBCs were removed. 200 ^{μl} of Chelex resin was added to the washed residue, boiled for 10 minutes and centrifuged. 10 ^{μl} of the supernatant thus obtained was used for the further amplification procedure.

The steps of boiling for ten minutes and subsequent refrigeration may be repeated.

DNA Amplification

The materials shown in Table 1 below were mixed and the mixture was subjected to DNA amplification procedure under the conditions described in Table 2 below using a PCR apparatus (Inno-lipa Co., Ltd. HLA Genotyping kit).

TABLE 1
Amplification Mixture (50 μl in total)

| | |
|---|---|
| Amplification buffer | 10 μl (dATP mix contained) |

| | | |
|---|---|---|
| Primer Mixture | 10 | μl |
| MgCl₂ | 10 | μl |
| Taq polymerase | 0.6 | μl |
| Extracted DAN | 5.0 | μl |
| dd H₂O | 14.4 | μl |

TABLE 2
PCR Conditions

| | | |
|---|---|---|
| 95° C. | 5 min. | 1 cycle |
| 95° C. | 20 min. | 30 cycles |
| 55° C. | 20 min. | |
| 72° C. | 30 min. | |
| 72° C. | 10 min. | 1 cycle |

Pre-treatment

1. Preparation of HLA DNA band strips

HLA DNA band strips were prepared from the amplified DNA using HLA kit (Inno-lipa Co., Ltd)

2. Preparation of the DNA solution to be injected

The DNA obtained through the amplification procedure was cloned into a vector by means of a TA cloning kit (Invitrogen Co., Ltd.). The cloned plasmids were separated in large amounts. E. coli HB101 was used, and the transformed E. coli HB101 was subjected to shaking incubation at 37° C. for 18 hours. The primarily HB101 was cultured overnight in a conventional LB (Luria Broth) medium. The cultured bacteria were diluted to 1/100 with 500 ^{μl} of LB, and then innoculated in the medium and cultured. Plasmids were separated by the method of Birnboin & Doly (1979). E. coli HB101 having the desired plasmid DNA was cultured at 37° C. for 18 hours. The reagents for separating the plasmids, Sol I [50 mM glucose, 25 mM Tris.Cl (pH 8.0), 10 mM EDTA(pH 8)], Sol II [0.2N NaOH, 1% SDS] and Sol III [5M potassium acetate 60 ^{ml}, glacial acetic acid 11.5 ^{ml}, H2O 28.5 ^{ml}] were added sequentially, and the mixture thus obtained gently stirred, and centrifuged at 14,000 rpm for 10 minutes. To the supernatant thus obtained was added an equivalent amount of phenol: chloroform (1:1), mixed vigorously, and then centrifuged. The step was repeated twice or three times. 100% ethanol (two fold) was added to the clear supernatant thus collected, and the mixture was refrigerated for 10 minutes and then centrifuged. The supernatant was discarded, and the residue was dried, washed once with 1 ^{ml} of 70% ethanol, dried, mixed with TE buffer solution to dissolve DNA, and refrigerated.

Glycerol and bromophenol blue were added to the DNA solution thus obtained.

Post-treatment

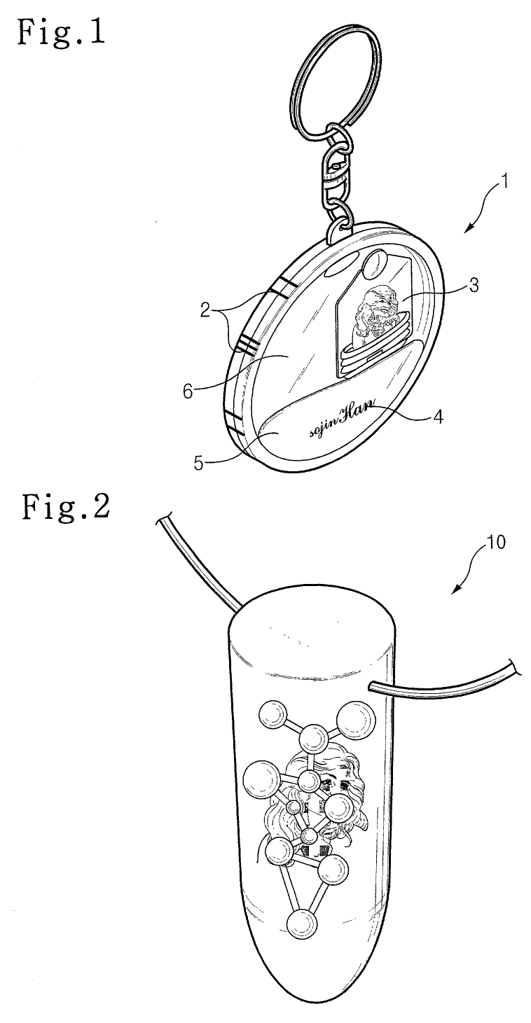

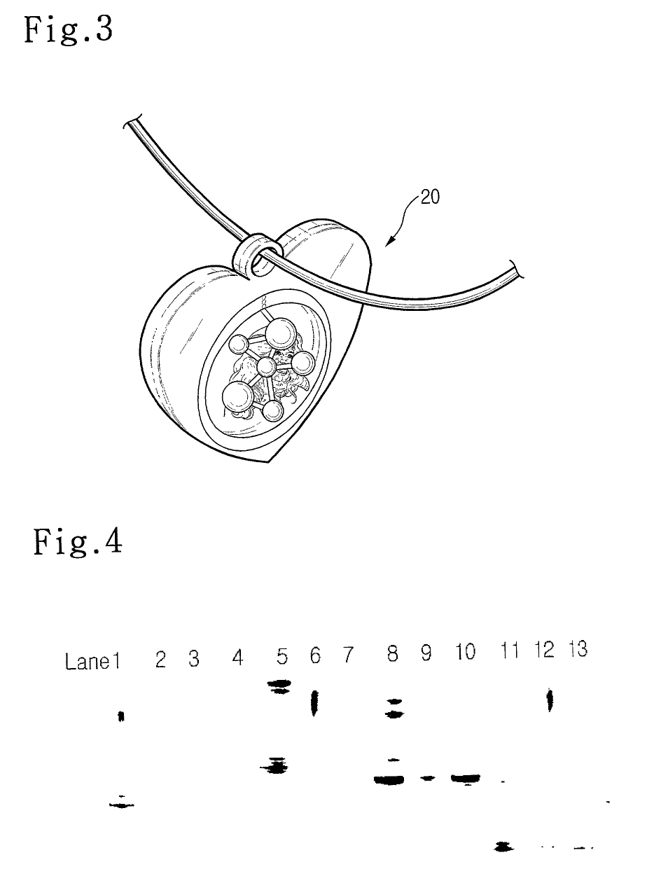

Two circular acrylic plates (each 50 m in diameter and 0.5 ^{cm} in thickness) were placed next to one another such that a cavity was formed between the contiguous surfaces of the plates. One of the plate has a hole which communicates with the cavity and through which the DNA solution may be injected into the cavity. The plate also has a member to which a ring may be connected. As shown in FIG. 1, HLA band strips were attached to the sides of the plates, and a picture and silk-screen signatures of the donor of the DNA were applied to the outer surfaces of the plate The acrylic plates touched to each other at their sides. The DNA solution prepared at the pre-treatment procedure was injected into the cavity through the hole in one of the plates, and the hole was then sealed. The acrylic object thus produced (1 cm in thickness) is symmetrical about a plane. A key ring was completed by inserting a ring to the ring connection member of the acrylic object.

### EXAMPLE 2

Extraction of DNA

Hair obtained from a donor was cut to within 5 cm from the roots. The hair was mixed with 200 ^{μl}of Chelex resin in a 5 ^{ml} tube, and the mixture was boiled for 10 minutes to remove cell debris other than DNA. The mixture was then centrifuged at 12,000 rpm. The supernatant thus obtained was used for the further amplification procedure.

DNA Amplification

The amplification mixture was prepared as shown in Table 3 below and then subjected to a DNA amplification procedure under the PCR conditions shown in Table 4 below.

TABLE 3
Amplification Mixture (50 μl in total)

| | | |
|---|---|---|
| Amplification buffer | 5 | μl |
| [Tris·Cl (pH 9.0), AMS, BSA, MgCl₂] | | |
| Primer Mixture | 4 | μl |
| [D17S695, D21S11, NerR, hTPO] | | |
| DNTP | 1 | μl |
| Taq polymerase (5 unit/ml) | 0.6 | μl |
| Extracted DAN | 5.0 | μl |
| dd H₂O | 34.4 | μl |

TABLE 4
PCR Conditions

| | | |
|---|---|---|
| 95° C. | 1 min. | 30 cycles |
| 62° C. | 1 min. | |
| 72° C. | 1 min. | |

Pre-treatment

1. Preparation of polyacrylamide gel film containing DNA

10% polyacrylamide gel was prepared and the amplified DNA was loaded. The DNA was subjected to electrophoresis by applying 200V to the electrophoresis column. After electrophoresis was completed, the gel was precipitated in 0.4% silver nitrate solution for 30 minutes in order to dye the DNA in the gel. The gel was placed between two sheets of cellophane paper and dried to yield a stiff gel film containing DNA. (1 mm in length, 3 cm in thickness)

2. Preparation of DNA solution to be injected

To obtain the DNA solution to be injected, phenol red and glycerol were added to the DNA obtained through the amplification procedure.

Post-treatment

A key ring was prepared according to the method of Example 1, except that the polyacrylamide gel containing DNA was used instead of HLA DNA band strips.

### EXAMPLE 3

Extraction and Preparation of DNA

Root portions of hairs taken from a DNA donor were cut into pieces 5 mm in size. The pieces carrying hair roots were put into a 1.5 ml tube and 200 ^{μl} of chelex resin was added, and the mixture was boiled for 10 minutes to remove cell debris except DNA. The mixture was then centrifuged at 12,000 rpm, and the supernatant was isolated and used for DNA amplification.

DNA Amplification

PCR was performed using 4 kinds of polymorphic markers, where the procedures were essentially the same as a normal PCR. The amplification mixture was prepared as shown in Table 5 below, and DNA amplification was carried out in a PCR apparatus (from MJ Research) under the conditions listed in Table 6 below.

TABLE 5
Amplification Mixture

| | | |
|---|---|---|
| Amplification buffer | 2.5 | μl |
| 10 mM dNTP | 0.5 | μl |
| Primer (10 pmol) | 2 | μl |
| Taq. DNA polymerase (5 units/μl) | 0.3 | μl |
| Extracted DNA | 5 | μl |
| ddH₂O | 14.7 | μl |
| Sum | 25 | μl |

TABLE 6
PCR Conditions

| | | |
|---|---|---|
| 50° C. | 3 min. | 1 cycle |
| 94° C. | 3 min. | 1 cycle |
| 94° C. | 30 sec. | 30 cycles |
| 62° C. | 30 sec. | |
| 72° C. | 30 sec. | |
| 72° C. | 5 min. | 1 cycle |

PCR products from amplification with the 4 kinds of polymorphic markers listed in Table 7 below were electrophoresed (8% polyacrylamide gel: 5% glycerol, 1×TBE, 0.1% TEMED, 0.8% ammonium persulfate, 50 mA, 200 V). After the electrophoresis, the gel was silver stained by fixing (10% acetic acid, 20 min.), staining (0.1% AgNO₃, 0.018% formaldehyde, 30 min.), and developing (3% sodium carbonate, 0.018% formaldehyde, 0.0002% sodium thiosulfate, 30 min.). The readout of the silver-stained gel was carried out as described below. The size of the DNA fragments in the stained gel was determined with reference to a marker of 20 bp DNA in size and previously typed PCR products.

TABLE 7
Reference Size of the 4 Kinds of Polymorphic Markers

| Polymorphic | Number of | Size of the |
|---|---|---|
| marker | repeating core | PCR product |
| D17S695 | 13 | 201 |
| D21S11 | 10 | 206 |
| HTPO | 11 | 217 |
| Humthol | 9 | 158 |

If the number of repeating core is greater than 10, the number minus 10 was used for the identification. For example, if the number was 13, 3 was used, and for 10, 0 was used. D17S695, D21S11, hTPO and humthol were listed in this order. Each marker was designated with a 2 digit number and the smaller number was listed first: 0/10 was expressed as 90, and 10/14 as 04.

### EXAMPLE 4

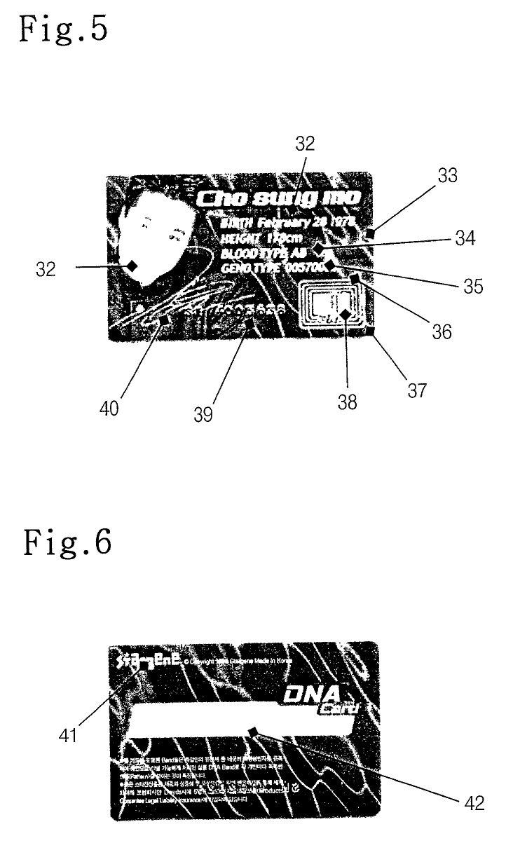

A picture 31 of a celebrity was printed on the left corner of a plastic card, and the name 32, date of birth 33, height 34, blood type 35, and genotype 36 of the celebrity were printed on the upper right corner (see. FIG. 5). Down the corner, a DNA bands image 38 (size: 0.6×1.0 cm) was printed according to the same procedure as in Example 3, and the printed image was covered with a hologram foil 37 (size: 1.6×2.1 cm). At the bottom portion of the card, manufacturer's serial number 39 was included and a signature of the celebrity 40 was added. FIG. 6 shows the backside of the card, where the manufacturer's company logo (1) and a signature box (2) were provided on the top portion.

### EXPERIMENTAL EXAMPLE 1

The following procedures were performed to confirm whether the donor's DNA is actually contained in the DNA cards prepared in Examples 3 and 4. For identification of DNA, the DNA containing portion of the card of Example 3 was cut out using scissors and placed in distilled water for a predetermined time. Then, aliquots of the aqueous solution was removed and subjected to DNA amplification using the 4 polymorphic markers as in the procedures for genotyping. The amplified DNA was electrophoresed on a polyacrylamide gel, silver stained, and compared with a separately prepared control DNA to confirm the genuineness. The electrophoresis and silver staining were performed according to the same protocols as used in Example 3. FIG. 4 shows the results obtained when two randomly picked products were compared with previously prepared control DNA of donor A. Line 1 shows the D17S695 control DNA of donor A; line 2 shows the D17S695 PCR product of Product 1 produced according to Example 3; line 3 shows the D17S695 PCR product of Product 2 produced according to Example 3; line 4 shows blank; line 5 shows the D21S11 control DNA of donor A; line 6 shows the D21S11 PCR product of Product 1 produced according to Example 3; line 7 shows the D21S11 PCR product of Product 2 produced according to Example 3; line 8 shows the hTPO control DNA of donor A; line 9 shows the hTPO PCR product of Product 1 produced according to Example 3; line 10 shows the hTPO PCR product of Product 2 produced according to Example 3; line 11 shows the humthol control DNA of donor A; line 12 shows the humthol PCR product of Product 1 produced according to Example 3; and line 13 shows the humthol PCR product of Product 2 produced according to Example 3.

As is seen in FIG. 4, DNA bands appear in the two products at the same location as in the control DNAs. The location of these DNA bands confirmed the genuineness.

What is claimed is:

1. A method for producing an object containing DNA, comprising the steps of:
image-scanning a polyacrylamide gel film containing electrophoresed DNA bands;
offset-printing on an object having a fixed shape using the scanned image data; and
applying a DNA solution onto the DNA band image which has been offset-printed.

2. The method according to claim 1, characterized in that the polyacrylamide gel film containing electrophoresed DNA bands is obtained by subjecting DNA which has been amplified using 4 kinds of polymorphic markers to an electrophoresis on a polyacrylamide gel, staining the gel and drying the stained gel.

3. The method according to claim 1 or 2, characterized in that the object is an object of toy, accessory, decorations, records, stationery, packaging papers, packages, sports gears, shoes, bags, clothes, cards, photos, souvenirs, art, funeral implements, wedding implements, or religious ceremony.

4. The method according to claim 3, characterized in that the object carries an identification of genotype.

5. An object containing DNA prepared by a method according to any one of claims 1 to 4.
